(19) United States
(12) Patent Application Publication
(10) Pub. No.: US 2018/0133029 A1
(43) Pub. Date: May 17, 2018

(54) METHOD FOR CONTROLLING AN ARTIFICIAL KNEE JOINT

(71) Applicant: OTTO BOCK HEALTHCARE PRODUCTS GMBH, Wien (AT)

(72) Inventor: Dirk SEIFERT, Wien (AT)

(73) Assignee: OTTO BOCK HEALTHCARE PRODUCTS GMBH, Wien (AT)

(21) Appl. No.: 15/569,054

(22) PCT Filed: Apr. 15, 2016
(86) PCT No.: PCT/EP2016/058406

§ 371 Date: Oct. 24, 2017

(30) Foreign Application Priority Data
Apr. 24, 2015 (DE) 10 2015 106 389.1

(51) Int. Cl. A61F 2/68; A61F 2/64; A61F 2/66

(52) U.S. Cl. CPC A61F 2/68; A61F 2002/5033; A61F 2/6607; A61F 2/64

(57) ABSTRACT

A method for controlling an artificial knee joint comprising an upper part and a lower part pivotally connected to each other, a resistance unit arranged between the upper part and the lower part and having an adjusting device to adjust the damping resistance, a control unit, the adjustment taking place on the basis of sensor data from at least one sensor. During the swing phase at least one of the knee angle (KA), the knee angle velocity (KAV), the knee angle acceleration (KAA), the lower limb angle, the lower limb velocity, the lower limb acceleration, the ankle moment (AM) and the axial force (AF) is sensed, the curve of the parameter is determined and the damping resistance is changed when, after an extreme value of the parameter is reached, the monotonic behavior of the curve of the parameter changes within the swing phase.

The invention relates to a method for controlling an artificial knee joint, in particular a prosthetic knee joint, having an upper part and a lower part which are fastened to one another in a manner pivotable about a pivot axis and having a resistance unit which is arranged between the upper part and the lower part and which has an adjustment device by means of which the damping resistance can be varied, having a control unit which is coupled to the adjustment device and which is connected at least to a sensor, wherein the adjustment is carried out on the basis of sensor data. The method is likewise usable for controlling orthosis knee joints or exoskeleton knee joints, and provided to this end.

Knee joints for orthoses, exoskeletons or prostheses have an upper part with an upper connection part and a lower part with a lower connection part, which are connected to one another in an articulated manner. As a rule, receptacles for a thigh stump or a thigh brace are arranged on the upper connection part, whereas a below-knee shaft or a below-knee brace is arranged on the lower connection part. In the simplest case, the upper part and the lower part are connected pivotably to one another by means of a uniaxial joint.

In order to be able to represent or support different requirements during the different phases of a step or during other movements or actions in a way that is as natural as possible, a resistance device is often provided which provides flexion resistance and extension resistance. The flexion resistance is used for setting how easily the lower part can be swung backwards in relation to the upper part when a force is applied. The extension resistance brakes the forward movement of the lower part and forms, inter alia, an extension limit stop.

DE 10 2008 008 284 A1 has disclosed an orthopedic knee joint with an upper part and with a lower part arranged pivotably thereon, which lower part is assigned a plurality of sensors, for example a flexion angle sensor, an acceleration sensor, an inclination sensor and/or a force sensor. The position of the extension stop is determined in a manner dependent on the sensor data.

DE 10 2006 021 802 A1 describes control of a passive prosthetic knee joint with adjustable damping in a flexion direction for adaptation of a prosthesis device with top-side connection means and with a connecting element to an artificial foot. The adaptation is made to climbing stairs, where a low-moment lifting of the prosthetic foot is detected, and the flexion damping, in a lifting phase, is lowered to below a level suitable for walking on a level surface. The flexion damping may be increased in a manner dependent on the change in the knee angle and in a manner dependent on the axial force acting on the lower leg.

DE 10 2009 052 887 A1 describes, inter alia, a method for controlling an orthotic or prosthetic joint with a resistance device and with sensors, wherein items of state information are provided by means of sensors during the use of the joint. The sensors detect moments or forces, wherein the sensor data of at least two of the determined variables are linked to one another by means of a mathematical operation, and in this way an auxiliary variable is calculated which is used as a basis for the control of the flexion and/or extension resistance.

According to the prior art, for the control of the change in the damping behavior, the sensor data are evaluated quantitatively, that is to say, as a rule, certain threshold values are predefined, in the case of the attainment or non-attainment of which the actuator is activated or deactivated, such that the resistance device provides an increased or reduced flexion or extension resistance.

It is an object of the present invention to provide a method in which problem situations when walking are identified in good time and in which there can be a quick reaction thereto by way of suitable adaptations of the resistance.

According to the invention, said object is achieved by means of a method having the features of the main claim. Advantageous embodiments and developments of the invention are disclosed in the dependent claims, and also in the description and the figures.

The method for controlling an artificial knee joint having an upper part and a lower part which are fastened to one another in a manner pivotable about a pivot axis and having a resistance unit which is arranged between the upper part and the lower part and which has an adjustment device by means of which the damping resistance can be varied, and having a control unit which is coupled to the adjustment device and which is connected at least to a sensor, wherein the adjustment is carried out on the basis of sensor data, provides for, during the swing phase, at least one characteristic from the group containing the knee angle, the knee angle velocity, the knee angle acceleration, the below-knee angle, the below-knee velocity, the below-knee acceleration, the ankle moment and the axial load to be captured, the profile of the characteristic to be determined and the damping resistance to be varied if, after reaching an extremum of the characteristic, the monotonic behavior of the profile of the characteristic changes within the swing phase, for example if, after attaining an extremum of the characteristic, the profile of the characteristic increases again within the swing phase. The method according to the invention is suitable, in particular, for recognizing deviations within a usual sequence of motion, in particular for recognizing a deviation from walking on a level surface or on ramps or other periodically recurring, uniform movement patterns which preferably have, at least in sections, a characteristic monotonic behavior. To this end, the below-knee and/or angle profile, the first and second time derivative of same and, optionally, the axial load on the lower part and, where applicable, the ankle moment are monitored. If a characteristic profile that deviates from the usual profile of the characteristics is recognized after reaching an extremum, for example a local maximum or a local minimum, during the swing phase which, for example, can be recognized by the absence of an ankle moment or an axial force that acts on the lower part along the below-knee tube or a below-knee brace, provision is made for the damping resistance to be varied such that the deviation from the known movement patterns is taken as a trigger signal for the damping resistance to be varied, for example by virtue of the damping resistance being increased or decreased. The change in the monotonic behavior is the indicator for the occurrence of a behavior that deviates from the usual gait pattern, e.g. interference by an obstacle. Here, the change in the monotonic behavior may consist both of a drop and of an increase in the characteristic profile. If a certain characteristic profile is expected for a certain duration or a certain angle range after an extremum and if there is a change in the monotonic behavior of the characteristic profile within this time or position range, then this is used as a trigger for varying the damping resistance. The method is provided for the control both of prostheses and of orthoses and exoskeletons. Where orthoses are referred to below, the explanations likewise apply to the special form of the orthosis in the form of an exoskeleton.

The deviation of certain characteristic profiles after reaching an extremum, in particular a local extremum, is based on a specific walking situation or specific circumstances during the swing phase; in particular, the assumption can be made that the foot, be this the prosthetic foot or the natural foot in the case of an orthosis, impacts on an obstacle, and so there is the risk of stumbling. In such a situation, it may be advantageous for the artificial knee joint to be secured against strong flexing in order to put the user of the artificial knee joint into a position where the artificial knee joint can be used to absorb the movement and the body weight such that the damping resistance against flexion is increased.

Advantageously, the damping resistance is increased to a stance phase damping level or therebeyond. The stance phase damping level is set for the respective patient, their weight and their level of activity and, as a consequence, individually adapted. In the stance phase, a flexion damping level that is greater than during the normal swing phase is provided as flexion of the artificial knee joint is desired during the swing phase so as to facilitate the foot or prosthetic foot swinging through to the front for impact of the heel. As a consequence, the damping resistance is increased to a stance phase damping level or therebeyond when a specific gait pattern is recognized in order to avoid flexion or make flexion more difficult in comparison with the swing phase.

Advantageously, a maximum is used as an extremum for the below-knee angle, the below-knee velocity, the below-knee acceleration, the knee angle, the knee angle velocity or the knee angle acceleration. The knee angle profile for normal walking and walking along ramps has a bell-shaped or approximately bell-shaped profile with a local maximum. The knee angle velocity has an approximately sinusoidal profile with a local maximum at the start of the swing phase and a local minimum at the end of the swing phase. The knee angle acceleration has a profile similar to that of the knee angle velocity but with a slight phase offset. The load on the lower part, be this due to an ankle moment or an axial force, drops to 0 after “toe off” has occurred. An external axial force is no longer applied after the foot has been lifted; an ankle moment is likewise absent. The local maximum characteristic profiles lie successive in time and within the scope of the swing phase flexion, at least for the knee angle velocity and the knee angle. The knee angle acceleration has its maximum value within the scope of the initial swing phase or at the end of the so-called pre-swing phase, and so it is possible to provide reliable monitoring of the gait behavior and a recognition of the risk of stumbling by way of the method when all characteristics that can be derived from the knee angle, possibly together with the ankle moment and axial force, are taken into account.

The below-knee angle, which is the angle of the lower leg in relation to the vertical direction, and the characteristics of the below-knee velocity and below-knee acceleration which are derived therefrom, also have a sinusoidal profile with an extremum when reaching the maximum knee angle and an extremum directed in the opposite sense upon heel strike. The damping resistance is varied in the case of disturbances of the profile of one or more characteristics that are related to the lower leg or the lower leg position in space. The position of the lower leg and hence the below-knee angle can be captured by way of an inertial angle sensor; the angle velocity or angle acceleration can be calculated by way of differentiation with respect to time.

In addition to an increase in the flexion resistance, which is often required, for facilitating a load on the joint, the reverse path also may be an expedient option for increasing the safety of the patient. To this end, the damping resistance against flexion in the swing phase is lowered to a level below a normal swing phase resistance if the ankle moment or the axial force, i.e. the load on the lower part, have dropped below a threshold or equal zero and the knee angle, the knee angle velocity and/or the knee angle acceleration experience a change in the monotonic behavior after reaching an extremum, for example rise again after reaching a maximum and a brief phase of dropping or drop again after reaching a minimum and a brief phase of increase. The threshold for detecting the load on the lower part lies close to complete unloading. Such an “elevating” strategy is expedient if a further swing through of the lower part and hence an increase in the distance of the foot from the floor are necessary.

Advantageously, a threshold a is set for the characteristics and, in particular, for the increase of the characteristic profile over an expected value after reaching an extremum, said threshold having to be exceeded so that the damping resistance is varied. Only after the threshold has been reached or the threshold has been exceeded is there an intervention in the conventional, pre-set resistance profile in order to prevent unwanted changes in the damping from already being undertaken in the case of minor deviations from the sequence of motion. This increases the tolerance of movement deviations and signal noise, without risking the safety of the patient.

Furthermore, it is possible to set a time threshold T which needs to be exceeded after reaching the extremum so that the damping resistance is varied. This ensures that an extremum is in fact present and that this is not only a slight deviation of the characteristic profile from the usual profile.

The profile of the characteristics is monitored permanently with a high sampling rate; the extrema of the characteristics advantageously are ascertained in real time in order to recognize disturbances in the sequence of motion in a timely manner and react in very good time with the correction measures in view of the damping resistance. Variations of the damping resistance on account of the above-described correction mechanism can be registered and stored in a memory such that a statistical evaluation of the interventions and corrections is possible. This allows statistical verification of the functionality and the usefulness of the employed method.

In particular, the advantage of the method can be seen in that it is able to operate independently of individual settings for the patient. All that is ascertained are relative relationships between the respective characteristic profiles; the weight, the gait speed or the like are irrelevant for detecting gait pattern deviations, for example stumbling. All that is used is an intrinsic pattern of the movement, making the method safe, insusceptible to disturbances and usable in a multifaceted manner.

The method is particularly suitable for patients who walk slowly, who have a greater risk of catching when walking and the stumbling connected therewith.

A development of the invention provides for those changes in the monotonic behavior that occur during the course of an unimpeded swing phase, for example after reaching extrema, in particular absolute extrema, not to be taken into account. A plurality of extrema may occur for a characteristic during the swing phase, said extrema each having a change in the monotonic behavior of the characteristic profile as a consequence. These changes in the monotonic profile should only be taken into account for changing the damping behavior if they would not occur, in any case, within the scope of the normal swing phase profile, i.e. for the knee or below-knee profile between the absolute maximum and the absolute minimum of the characteristic profiles; the same also applies to the respective first derivatives of these characteristics with respect to time.

The resistance unit may for example be configured as an actuator, for example as a hydraulic, pneumatic, magnetorheological, magnetic, electrical, mechanical or electromagnetic resistance unit. In the case of hydraulic or pneumatic resistance units, flow transfer channels are closed, such that said flow transfer channels can no longer allow medium to flow from an extension chamber into a flexion chamber. In this way, the flow of the medium between the extension chamber and the flexion chamber can possibly also be prevented entirely. In the case of mechanical resistance devices, it is for example the case that the friction is increased to such an extent that no further flexion is possible. The same applies to electrically actuated resistance units.

Use may also be made of actuators which both actively introduce energy into the system and also conversely extract energy from the system, and thereby act as a resistance unit. Actuators may for example be formed as electric motors, hydraulic or pneumatic pumps or piezoelectric elements.

An exemplary embodiment of the invention will be discussed in more detail below on the basis of the appended figures. In the figures:

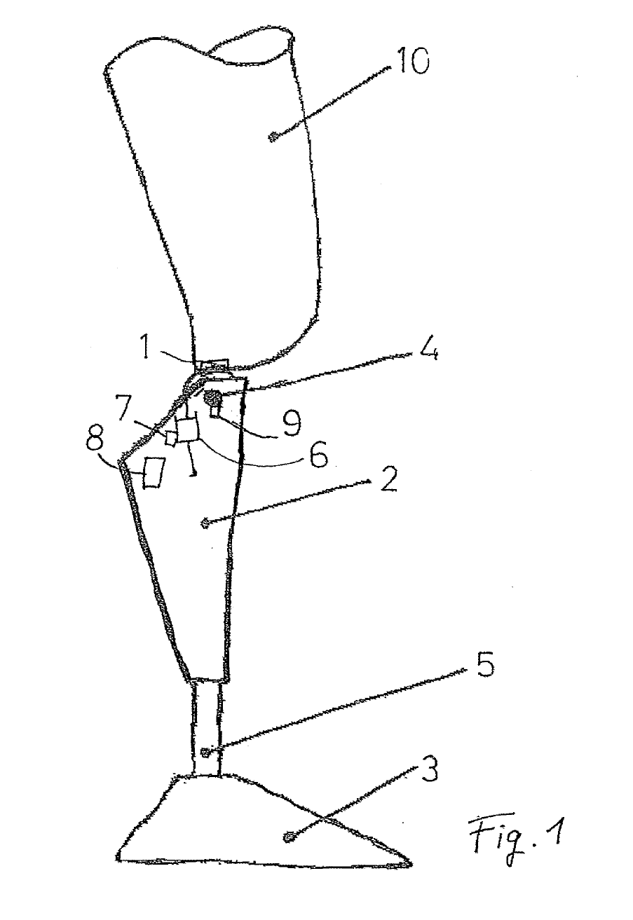

FIG. 1—shows a schematic illustration of a prosthesis,

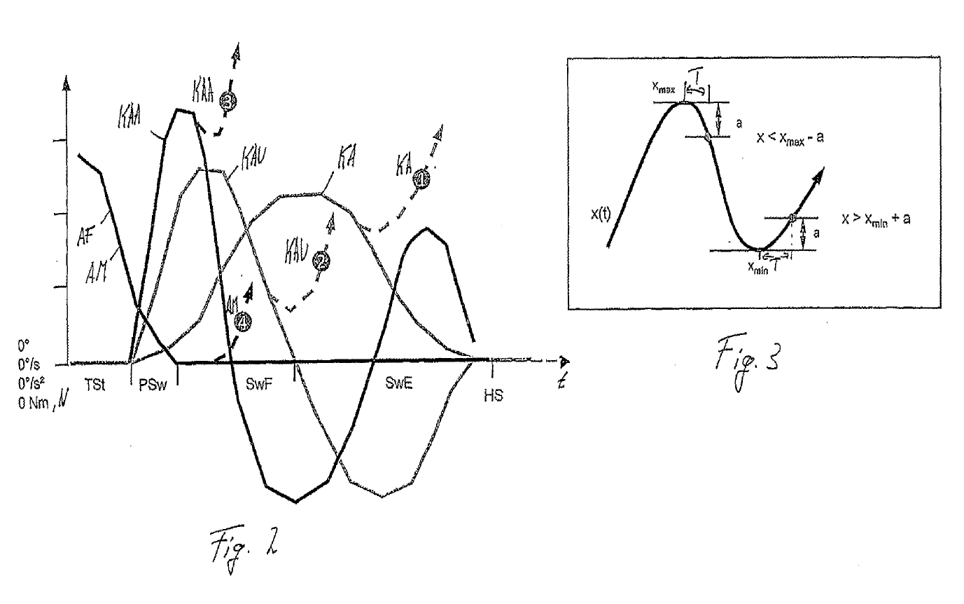

FIG. 2—shows a schematic profile of the characteristics; and

FIG. 3—shows a schematic illustration of taking a threshold into account.

FIG. 1 shows, in a schematic illustration, a leg prosthesis with an upper part 1 to which a thigh socket 10 for receiving a thigh stump is fastened. A lower part 2 designed as a lower leg part is arranged pivotably on the upper part 1. The lower part 2 is mounted on the upper part 1 pivotably about a pivot axis 4. The lower part 2 has a lower leg tube 5, to the distal end of which there is fastened a prosthetic foot in which there may be accommodated a device for determining the axial force acting on the lower leg tube 5 and the ankle moment acting about the fastening point of the prosthetic foot 3 to the lower leg tube 5.

In or on the lower part 2 there is arranged a resistance device 6 which may be formed for example as a damper or actuator and which is supported between the upper part 1 and the lower part 2 in order to provide an adjustable extension resistance and flexion resistance. The resistance device 6 is assigned an adjustment device 7, for example a motor, a magnet or some other actuator, by means of which the respective resistance within the resistance device 6 can be varied. If the resistance device 6 is formed as a hydraulic damper, it is possible by means of the adjustment device 7 for the respective flow cross section to be increased or decreased in size. This may be realized by opening or closing valves or changing viscosities or magnetorheological properties. If the resistance device is formed as an electric motor operating as a generator, it is possible for an increase or decrease in the respective resistances to flexion or extension to be set through variation of the electrical resistance.

To be able to activate or deactivate the adjustment device 7, a control device 8 is assigned to the lower part 2, in particular is accommodated in a lower leg cover, by means of which control device a corresponding activation or deactivation signal is output to the adjustment device 7. The adjustment device 7 is activated or deactivated on the basis of sensor data, and the sensor data are provided by one or more sensors 9 which are arranged on the artificial knee joint. These may be angle sensors, acceleration sensors and/or force sensors. The sensors 9 are connected to the control device 8, for example by cable or by means of a wireless transmission device.

The entire step cycle from the heel strike to the new, next heel strike HS, and thus also the entire swing phase with the swing phase extension and the swing phase flexion, is monitored by means of the sensors 9.

In order to control the damping by way of the resistance device 6, it is the knee angle profile and the first and second derivative thereof with respect to time that are monitored in particular. The knee angle KA is plotted in FIG. 2 over time. Likewise, the profile of the knee angle velocity KAV and the knee angle acceleration KAA are illustrated over time in FIG. 2. Moreover, the diagram illustrates the load in the form of an axial force AF and ankle moment AM, which substantially correspond in terms of their profile such that both load variables AF, AM are illustrated using a single curve.

There is a reduction in the axial load AF and in the ankle moment AM as well within the scope of the terminal stance phase TSt; the knee angle KA, the knee angle velocity KAV and the knee angle acceleration KAA increase after completion of the terminal stance phase TSt. The profile of the knee angle KA is substantially bell-shaped and increases, up to a knee angle maximum, until the end of the swing phase flexion SwF in order then to reduce in the swing phase extension phase SwE, likewise in a substantially bell-shaped manner, until the heel strike HS and to be present upon heel strike HS in the maximally extended position, in which the knee angle KA is assumed to be zero degrees.

The knee angle velocity KAV has a substantially sinusoidal profile. A comparatively quick rise in the knee angle velocity KAV can be determined at the start of the swing phase, i.e. in the so-called pre-swing phase PSw; after reaching a local maximum, the knee angle velocity drops to zero when a maximum knee angle KA is reached, becomes negative, reaches a relative minimum and then increases again up to the heel strike HS in order to be zero when a maximum extended position is reached.

The knee angle acceleration KAA reaches its relative maximum at the end of the pre-swing phase PSw before the maximum of the knee angle velocity KAV, it has a first zero crossing when the maximum knee angle velocity KAV is reached, reaches a minimum in the region of the zero crossing of the knee angle velocity KAV and reaches a second relative maximum at the end of the swing phase extension.

The knee angle profile, knee angle velocity profile and knee angle acceleration profile illustrated thus respectively occur when there is unimpeded walking. A disturbance, for example stumbling, can be assumed if the knee angle KA has reached a local maximum and dropped below it again, and subsequently increases again, as illustrated by the dashed line KA₁ in FIG. 2.

Furthermore, a disturbance or stumbling can be assumed if, after reaching a local maximum of the knee angle velocity KAV and after a decrease in the knee angle velocity KAV, the latter increases again within the swing phase, as illustrated by the curve profile KAV₂. Likewise, stumbling can be assumed if the knee angle acceleration KAA reduces again after reaching a local maximum and subsequently increases again, as illustrated by the curve profile KAA₃.

The respective maxima of the curve profiles are determined in real time. In order to be able to tolerate the signal noise or slight movement deviations, thresholds a may be defined for the drop below the respective maxima and the increase in the curve profile or of the sensor signal.

FIG. 3 schematically illustrates a characteristic profile. The damping setting is not varied for as long as the threshold a is not exceeded, i.e. a deviation within a certain time threshold T is not sufficiently large. If the characteristic profile exceeds a pre-determined threshold a within a predetermined time interval T, the assumption is made on the part of the controller that stumbling is present, for example that the foot catches when walking or impacts on an obstacle with the heel, in order then to undertake a variation in the damping.

The damping usually is increased; the level of increase can vary but the stance phase damping level is usually set to be higher, although this is not mandatory.

In order to decide whether a reduction or an increase in the flexion resistance occurs, it is possible to take into account a load on the lower part 2, for example by way of the occurrence of an ankle moment AM. If an axial force AF occurs during a normally occurring time interval during the walking, a termination of the swing phase and, hence, a disturbance in the gait pattern can be expected, and so an increase or decrease in the flexion damping is indicated. If an axial force component occurs in the distal direction, a further swing through of the prosthetic foot may be necessary, and so reduction in the flexion resistance is advantageous and set accordingly.

In addition to the knee angle KA and the derivatives thereof with respect to time, it is also possible to use the load on the lower part during the swing phase, i.e. an axial force AF or an ankle moment AM, for control purposes. After reaching a minimum after the toe off with an axial force AF or an ankle moment AM of zero, each increase in the axial force AF or in the ankle moment AM within a normally provided time interval until the heel strike HS is identified as a disturbance which leads to a change in the damper settings.

1. A method for controlling an artificial knee joint, the method comprising:
providing an upper part and a lower part which are pivotally connected to each other about a pivot axis, a resistance unit which is arranged between the upper part and the lower part and which has an adjustment device to vary the damping resistance, a control unit which is coupled to the adjustment device and which is connected to at least one sensor, the adjustment being carried out using sensor data from the at least one sensor;
capturing during a swing phase, at least one characteristic from a knee angle, a knee angle velocity, a knee angle acceleration, a below-knee angle, a below-knee velocity, a below-knee acceleration, an ankle moment, and an axial load;
determining a profile of the characteristic;
varying the damping resistance if, after reaching an extremum of the characteristic, a monotonic behavior of the profile of the characteristic changes within the swing phase.

2. The method as claimed in claim 1, wherein the damping resistance is increased.

3. The method as claimed in claim 2, wherein the damping resistance is increased to a level of the stance phase damping or therebeyond.

4. The method as claimed in claim 1, wherein a maximum is used as an extremum for the below-knee angle, the below-knee velocity, the below-knee acceleration, the knee angle, the knee angle velocity or the knee angle acceleration.

5. The method as claimed in claim 1, wherein the damping resistance in the swing phase is lowered to a level below a swing phase standard resistance if an ankle moment or an axial force are zero or have dropped below a threshold and at least one of the knee angle, the knee angle velocity, and the knee angle acceleration changes in the monotonic behavior after reaching the extremum.

6. The method as claimed in claim 1, wherein a threshold that needs to be exceeded so that the damping resistance is varied is set for the characteristics.

7. The method as claimed in claim 1, wherein a time threshold that needs to be exceeded after reaching the extremum so that the damping resistance is varied is set.

8. The method as claimed in claim 1, wherein the extrema of the characteristics are ascertained in real time.

9. The method as claimed in claim 1, wherein variations of the damping resistance are registered and stored in a memory.

10. The method as claimed in claim 1, wherein changes in the monotonic behavior that occur during the course of an unimpeded swing phase are not taken into account.

11. A method for controlling an artificial knee joint, the method comprising:
providing an upper part and a lower part, a damping device, and a control unit., the upper and lower parts being pivotally connected to each other, the resistance unit having an adjustment device to adjust the damping resistance, the control unit being coupled to the adjustment device and at least one sensor and operable to adjust the damping resistance using sensor data from the at least one sensor;
capturing, during a swing phase, at least one characteristic from at least one of a knee angle, a knee angle velocity, a knee angle acceleration, a below-knee angle, a below-knee velocity, a below-knee acceleration, an ankle moment, and an axial load;
determining a profile of the characteristic;
varying the damping resistance if a monotonic behavior of the profile of the characteristic changes within the swing phase after reaching an extremum of the characteristic.

12. The method as claimed in claim 11, wherein varying the damping resistance includes increasing the damping resistance.

13. The method as claimed in claim 12, wherein increasing the damping resistance includes increasing the damping resistance to at least a level of a stance phase damping.

14. The method as claimed in claim 11, wherein a maximum is used as an extremum for the below-knee angle, the below-knee velocity, the below-knee acceleration, the knee angle, the knee angle velocity or the knee angle acceleration.

15. The method as claimed in claim 11, further comprising lowering the damping resistance in the swing phase to a level below a swing phase resistance if an ankle moment or an axial force are zero or have dropped below a threshold and at least one of the knee angle, the knee angle velocity, and the knee angle acceleration changes in the monotonic behavior after reaching the extremum.

16. The method as claimed in claim 11, further comprising setting a threshold to be exceeded so that the damping resistance is varied for the characteristics.

17. The method as claimed in claim 11, further comprising setting a time threshold to be exceeded after reaching the extremum so that the damping resistance is varied.

18. The method as claimed in claim 11, further comprising ascertaining the extrema of the characteristics in real time.

19. The method as claimed in claim 11, further comprising registering variations of the damping resistance and storing the registered variations in a memory.

20. The method as claimed in claim 11, wherein changes in the monotonic behavior that occur during the course of an unimpeded swing phase are not taken into account.
